(19) United States
(12) Patent Application Publication
(10) Pub. No.: US 2015/0259305 A1
(43) Pub. Date: Sep. 17, 2015

(54) CATALYTIC REACTION

(71) Applicant: National Tsing Hua University, Hsinchu (TW)

(72) Inventors: Michael Hsuan-Yi HUANG, Hsinchu (TW); Sourav REJ, Hsinchu (TW); Kaushik CHANDA, Hsinchu (TW)

(21) Appl. No.: 14/320,407

(22) Filed: Jun. 30, 2014

(30) Foreign Application Priority Data
Mar. 11, 2014 (TW) 103108341

(51) Int. Cl. C07D 261/08; B01J 23/72; B01J 23/52; C07D 249/06

(52) U.S. Cl. CPC C07D 261/08; C07D 249/06; B01J 23/72; B01J 23/52

(57) ABSTRACT

A catalytic reaction comprises several steps: providing a catalyst, wherein the catalyst is metal or metal oxide particles and at least have {110} crystal plane; using the catalyst when performing a cycloaddition reaction. By using the catalyst with high reactivity, reaction rate is dramatically promoted.

### BACKGROUND OF THE INVENTION

1. Field of the Invention

The present invention is related to a catalytic reaction, particularly to a catalytic reaction provided by a nanocatalyst with {110} crystal plane.

2. Description of the Prior Art

Catalysts, which provide an easier and faster reaction pathway, may play a critical role in chemical reaction. Catalytic nanoparticles are often used for catalyzing cycloaddition reaction and promote the click reaction. Triazole is one of the products of the cycloaddition reaction, and compounds comprising triazole structure, such as antifungal drug and pesticides, are usually used in the research of biomedicine and biochemistry mechanism. In addition, catalytic nanoparticles may catalyze other organic reactions, for example, the cycloaddition reaction for synthesis of isoxazoles.

Catalytic nanoparticles of metal and metal oxide may comprise various type of metals based on user's requirement, take cuprous oxide (Cu₂O) and gold for example, Cu₂O and gold which are provided excellent electro-optical properties are easily prepared into nanoparticles and usually used for catalyzing chemical reactions, such as cycloaddition reaction, and other semiconductor applications. Besides, Cu₂O and gold are non-toxic and reactive in aqueous solution. In addition, Cu₂O and gold may be recyclable so as to provide a quicker reaction with lower cost due to the heterogeneous catalysis.

However, conventional metal or metal oxide catalysts still have limited catalytic effect. It is a major issue in current industry to discovery for catalysts with increased reaction rate and significantly improved yield.

### SUMMARY OF THE INVENTION

One of the objectives of the present invention is directed for providing a metal or metal oxide catalyst with {110} crystal plane so as to increase adsorption sites for reactant and reaction reactivity and improve the rate of cycloaddition reaction, so as to achieve higher yield.

According to an embodiment of the present invention, a catalytic reaction comprises: providing a catalyst, wherein the catalyst is made of metal or metal oxide nanoparticles and at least comprises {110} crystal plane; providing a first unsaturated compound and a second unsaturated compound; and providing the catalyst to perform a cycloaddition reaction of the first unsaturated compound and the second unsaturated compound and obtained a product.

The purpose, technical content, characteristic and effect of the present invention will be easy to understand by reference to the following detailed descriptions, when taken in conjunction with the accompanying drawings and the particular embodiment.

### BRIEF DESCRIPTION OF THE DRAWINGS

FIGS. 1 to 4 are the data of the embodiment of the present invention.

### DESCRIPTION OF THE PREFERRED EMBODIMENT

To achieve the major objective, an embodiment of the present invention provides a catalytic reaction, comprising steps of providing a catalyst, wherein the catalyst is made of metal or metal oxide nanoparticles and at least comprises {110} crystal plane; and the catalyst catalyzes an organic reaction. Here, the organic reaction may be a cycloaddition, a step thereof comprises providing a first unsaturated compound and a second unsaturated compound; and providing the said catalyst to perform a cycloaddition reaction of the first unsaturated compound and the second unsaturated compound so as to obtain a product.

There may be various categories of the cycloaddition reaction. Based on atom number participated in the reaction, cycloaddition reaction may be a [2+2] cycloaddition reaction, a [2+3] cycloaddition reaction, a [3+2] cycloaddition reaction, a [4+2] cycloaddition reaction, a [4+3] cycloaddition reaction, or a [6+4] cycloaddition reaction. In terms of reaction mechanisms to be classified, cycloaddition reaction may be Diels-Alder reaction, Huisgen cycloaddition reaction or Nitrone-olefin cycloaddition reaction.

In addition, it is known for those with ordinary skill in the art that transition metal catalysts (for example, Au, Ag, Cu, Pd, Ru, Ni) may be used for catalyzing various types of cycloaddition reactions, for example but not limited that, copper catalyst may catalyze Huisgen cycloaddition reaction or 1,3 bipolar cycloaddition reaction and the like; gold catalyst may catalyze [3+2] or [2+2] cycloaddition reaction; silver catalyst may catalyze [3+2] cycloaddition reaction and the like.

In the present invention, cycloaddition reaction may be achieved by ways of click reactions. Click chemistry, which is a quite important concept in modern chemistry, mainly uses the joining between small molecules to form various molecules. Originally, in many chemistry reactions, joining between different molecules requires precisely controlled parameters and complicated process. However, click reaction occurs between specific structures in different small molecules, and “click” implies simply snap-fit to each other, such as the connection between backpack buckle or latch, so as to provide many advantages such as simplicity for preparation, high yield, with almost no by-products and less interference from other functional groups. The occurrence of click reaction may be promoted by good regioselectivity and reactivity.

Crystal plane and shape of the catalyst are critical factors that affect selectivity. For gold nanoparticle, surface atomic density of different crystal panels {100}, {111} and {110} are 13.873, 12.015 and 8.496 atoms/nm², respectively, and unsaturated gold coordinate bond are 3, 4 and 5, respectively. It suggests that crystal panel {110} has higher degree of unsaturation, so that it may adsorb more specific reaction molecular structure in a faster manner so as to elevate reactivity with different reacting molecules. In particularly, the rhombic dodecahedra of gold nanoparticle in the present invention include much more crystal panels {110}, so as to obtain the better effect for catalysis. Cuprous oxide (Cu₂O) also has similar properties as above mentioned, and in comparison to crystal panels {100} and {111}, in which part of copper atoms are covered by oxygen atom, crystal panel {110} of Cu₂O, which exposes copper atoms completely, have higher activity. Especially, Cu₂O rhombic dodecahedra may have better catalytic property as well as regio selectivity.

As for further limited reactants, the first unsaturated compound and the second unsaturated compound may be selected from alkenes, alkynes and 1,3 bipolar compound. In an embodiment, alkynes is represented by formula (1): R₁ [symbol] . . . (1), wherein R1 is independently selected from a group consisting of hydroxyl, carboxyl, ester, nitro, alkyl silicon, substituted or unsubstituted C1-C10 alkyl, substituted or unsubstituted C2-C10 alkenyl, substituted or unsubstituted C2-C10 alkynyl, substituted or unsubstituted C3-C20 cycloalkyl, substituted or unsubstituted C3-C20 cycloalkenyl, substituted or unsubstituted C1-C20 heterocycloalkyl, substituted or unsubstituted C1-C20 heterocycloalkenyl, substituted or unsubstituted aryl, and substituted or unsubstituted heteroaryl.

In another embodiment, the said 1,3 dipolar compound comprises azides, wherein the azides is represented by formula (2): R₂—N₃ . . . (2), wherein R2 is independently selected from a group consisting of hydroxyl, carboxyl, ester, nitro, alkyl silicon, substituted or un substituted C1-C10 alkyl, substituted or unsubstituted C2-C10 alkenyl, substituted or unsubstituted C2-C10 alkynyl, substituted or unsubstituted C3-C20 cycloalkyl, substituted or unsubstituted C3-C20 cycloalkenyl, substituted or unsubstituted C1-C20 heterocycloalkyl, substituted or unsubstituted C1-C20 heterocycloalkenyl, substituted or unsubstituted aryl, and substituted or unsubstituted heteroaryl

Except the aforementioned azides, 1,3 dipolar compound further comprises oximes, wherein the oximes is represented by formula (3):

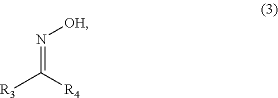

wherein R3 is independently selected from a group consisting of hydroxyl, carboxyl, ester, nitro, alkyl silicon, substituted or un substituted C1-C10 alkyl, substituted or unsubstituted C2-C10 alkenyl, substituted or unsubstituted C2-C10 alkynyl, substituted or unsubstituted C3-C20 cycloalkyl, substituted or unsubstituted C3-C20 cycloalkenyl, substituted or unsubstituted C1-C20 heterocycloalkyl, substituted or unsubstituted C1-C20 heterocycloalkenyl, substituted or unsubstituted aryl, and substituted or unsubstituted heteroaryl; R4 is selected from hydrogen and halogen.

Here, “aryl” refers to aromatic ring system including 6-simple carbon ring, 10-double carbon ring and 14-triple carbon ring. Examples of aryl moieties include phenyl (Ph), phenylene, naphthyl, naphthylene, pyrenyl, anthryl, and phenanthryl.

“Heteroaryl” may include a 5-8 membered single aromatic ring with 1-3 heteroatoms, 8-12 membered double aromatic rings with 1-6 heteroatoms or 11-14 membered triple aromatic rings with 1-9 heteroatoms. The aforementioned heteroatoms are selected from O, N or S (for example, single aromatic ring, double aromatic rings or triple aromatic rings include carbon atom and 1-3, 1-6 or 1-9 N, O or S heteroatoms, respectively).

Examples of heteroaryl moieties may include furyl, furylene, fluorenyl, pyrrolyl, thienyl, oxazolyl, imidazolyl, thiazolyl, pyridyl, pyrimidinyl, quinazolinyl, quinolyl, isoquinolyl and indolyl.

Products of the cycloaddition reaction may be provided in various forms, comprising heterocyclic compound or cyclic compound. Furthermore, the products may be triazole or isoxazoles.

In an embodiment, triazole is represented by formula (4):

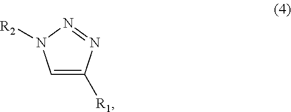

wherein each of R1 and R2 is independently selected from a group consisting of hydroxyl, carboxyl, ester, nitro, alkyl silicon, substituted or un substituted C1-C10 alkyl, substituted or unsubstituted C2-C10 alkenyl, substituted or unsubstituted C2-C10 alkynyl, substituted or unsubstituted C3-C20 cycloalkyl, substituted or unsubstituted C3-C20 cycloalkenyl, substituted or unsubstituted C1-C20 heterocycloalkyl, substituted or unsubstituted C1-C20 heterocycloalkenyl, substituted or unsubstituted aryl, and substituted or unsubstituted heteroaryl.

Isoxazoles is represented by formula (5):

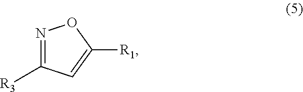

wherein R1 and R3 are independently selected from a group consisting of hydroxyl, carboxyl, ester, nitro, alkyl silicon, substituted or un substituted C1-C10 alkyl, substituted or unsubstituted C2-C10 alkenyl, substituted or unsubstituted C2-C10 alkynyl, substituted or unsubstituted C3-C20 cycloalkyl, substituted or unsubstituted C3-C20 cycloalkenyl, substituted or unsubstituted C1-C20 heterocycloalkyl, substituted or unsubstituted C1-C20 heterocycloalkenyl, substituted or unsubstituted aryl, and substituted or unsubstituted heteroaryl.

The shape, type and size of catalysts are described as follows. Catalysts may be cubes or octahedra, comprising perfect cubes, cubes with cut edge, perfect octahedra and octahedra with cut edge. During crystal growth, different crystal planes have different growth rates, and different crystal shapes may be formed resulting from competitive growth. Cubes and octahedra with perfect crystal plane represent crystal plane {100} and {111}, respectively, and cubes and octahedra with cut edge represent crystal plane {110}. In addition, catalyst may be rhombic dodecahedron, comprising perfect rhombic dodecahedron and rhombic dodecahedron with cut edge. Perfect rhombic dodecahedron represent integral crystal plane {110}. Although rhombic dodecahedron with cut edge still comprise crystal plane {110}, the ratio of the crystal panel decreases as the increases of the cut edge area.

Catalyst may be made of metal or metal oxide, such as Cu₂O or gold. The particle size of the catalyst ranges from 30-300 nm.

In addition to the organic reaction described above, the catalyst of the present invention may be widely applied to other organic reactions. Gold catalyst may catalyze oxidation of carbon monoxide, propylene epoxidation, hydrogenation of unsaturated hydrocarbons and the like and be applied to mouth masks, exhaust pipe of vehicle or exhaust outlet and the like. According to the metal or metal oxide catalyst with crystal panel {110}, reaction rate and yield of organic reaction mentioned above may significantly increase. The characteristics of the catalyst have been described above, and the repeated description will be omitted.

Some of the embodiments of the present invention are shown in paper of Chanda, K. et al. (“Investigation of Facet Effects on the Catalytic Activity of Cu₂O Nanocrystals for Efficient Regioselective Synthesis of 3,5-Disubstituted Isoxazoles”, Nanoscale 2013, 5, 12494; “Facet-Dependent Catalytic Activity of Cu₂O Nanocrystals in the One-Pot Synthesis of 1,2,3-Triazoles by Multicomponent Click Reactions”, Chem.—Eur. J. 2013, 18, 16036.), which is incorporated herein by reference.

The purpose, technical content, characteristic and effect of the present invention will be easy to understand by reference to the following detailed descriptions, when taken in conjunction with the accompanying drawings and the particular embodiment, but not for limit the scope of the present invention.

### Example 1

### Synthesis of Nanocubes and Dodecahedron Cu₂O

For the synthesis of Cu₂O nanocrystals with cubic and rhombic dodecahedral shapes, 8.92 and 6.9 ml of deionized water were added into sample vials, respectively. The volume of water added into each vials was adjusted in such a manner that after the addition of NH₂OH.HCl, the total volume of final solution is 10 ml. The sample vials was placed in water bath at 30-32° C. Then, a solution of CuCl₂ (0.5 ml, 0.1 M) and SDS powder (0.087 g) was added to the sample vials with vigorous stirring. When the solution become clear, a solution of NaOH (0.18 ml, 1.0 M) was added and shaken for 10 s. due to the precipitation of threadlike Cu(OH)₂, the solution becomes light blue immediately. Finally, 0.40 ml and 2.37 ml, 0.1 M of NH₂OH.HCl were quickly injected in 5 s to form nanocubes and dodecahedron, respectively. After stirring for 20 s, the solution was kept in water bath for 1 h to grow the nanocrystal. The concentration of Cu²⁺ ion and SDS surfactant in the final solution are 1.0×10⁻³ M and 3.0×10⁻² M, respectively. The reaction mixtures were centrifuged at 5000 rpm for 3 min. After pouring the top of solution, rinsed the residue with 6 ml of 1:1 volume ratio of ethanol to water three times to remove unreacted chemicals and SDS surfactant. In final wash step, 5 ml of ethanol was used to disperse the residue into 0.6 ml of ethanol for storage and analysis.

### Example 2

### Synthesis of Octahedra Cu₂O

Firstly, 9.02 ml of deionized water was added into sample vial. The sample vial was placed in water bath at 30-32° C. Next, 0.1 ml, 0.1 M of CuCl₂ and 0.2 ml, 0.1 M of NaOH solution was added, and the vial was shaken for 10 s. And then, 0.087 g of SDS powder was introduced with vigorous stirring. Finally, 0.68 ml, 0.2 M of NH₂OH.HCl was quickly injected. After stirring for 20 s., the solution was kept in the water bath for 2 h to grow the nanocrystals. The concentration of Cu²⁺ ion and SDS surfactant in the final solution are 1.0×10⁻³ M and 3.0×10⁻² M, respectively. The reaction mixture was centrifuged at 3500 rpm for 2 min. After pouring the top of solution, residue was rinsed with 6 ml of 1:1 volume ratio of ethanol to water three times, to remove unreacted chemicals and SDS surfactant. In final wash step, 5 ml of ethanol was used to disperse the residue into 0.6 ml of ethanol for storage and analysis.

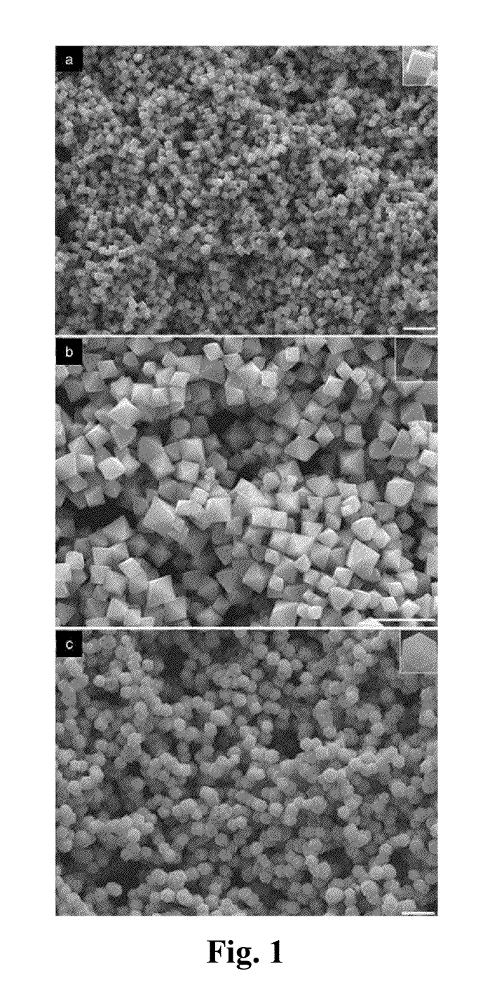

The photographs of cubes, octahedra Cu₂O and dodecahedron Cu₂O obtained from above steps are shown in (a), (b) and (c) of FIG. 1, wherein the scale equals to 1 μm.

### Example 3

### Cycloaddition Reaction Catalyzed by Using Nano Cu₂O to Form 1,2,3-Triazoles

First, first unsaturated compound and second unsaturated compound were provided. The first unsaturated compound is alkynes (such as formula (1)), R1 is phenyl, alkyl or hydroxyl. The second unsaturated compound R2-N₃ (such as formula (2)) may pre-synthesize by azide salts NaN₃ and bromide R2-Br. Then, 0.25 mmol of first unsaturated compound and 0.25 mmol of second unsaturated compound were placed in ethanol (or water) at 55° C. under nitrogen atmosphere to obtain 1,2,3-Triazoles. Or, the first unsaturated compound and azide salts NaN₃ and bromide R2-Br were placed into ethanol (or water) simultaneously and reacting at same time to obtain 1,2,3-Triazoles.

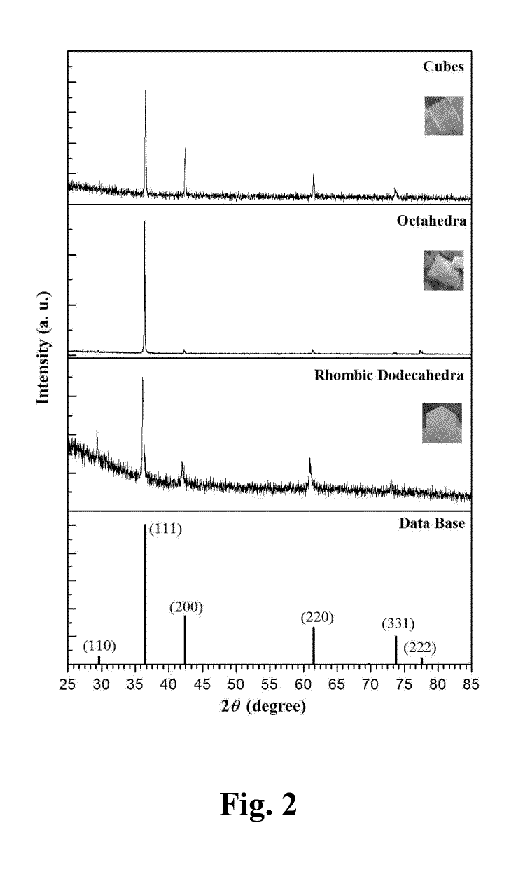

In order to detect the catalytic effect of various catalysts, cubes, octahedra (OC) or rhombic dodecahedra (RD) of the nano Cu₂O particles are added into the reaction. FIG. 2 shows X-ray diffraction patterns of nano Cu₂O particles, the comparisons of reaction time/yield are shown in table 1 below. Rhombic dodecahedra (RD) of the nano Cu₂O particles catalyst reaches the fastest reaction rate in three reactions listed in Table 1, due to the highest crystal panel {110} ratio thereof.

TABLE 1
Comparison of reaction time and yield of forming 1,2,3-Triazoles by cycloaddition reaction catalyzed by different shapes of nano Cu₂O

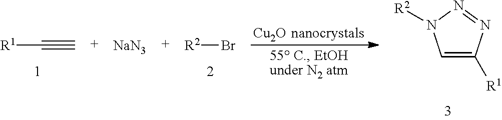

| | | | | Time (h)/ | Time (h)/ | Time (h)/ |
|---|---|---|---|---|---|---|
| Num- | | Organic | | Yield ^{b} | Yield ^{b} | Yield ^{b} |
| ber | Alkynes | Halides | Product | (rd) | (oc) | (cube) |
| 1 | 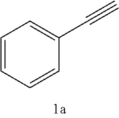 | 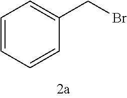 | 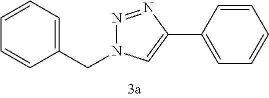 [SMILES: c1ccc(Cn2cc(-c3ccccc3)nn2)cc1] | 1/96 | 4.5/90 | 7/88 |
| 2 | 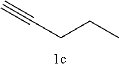 | 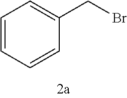 | 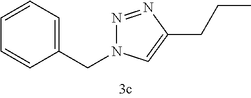 | 1.5/92 | 5/88 | 7/80 |
| 3 | 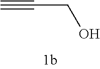 | 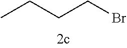 | 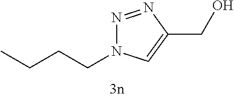 | 2/90 | 5.5/90 | 8/77 |

^{a} Reagents and conditions: 1 (0.25 mmol), 2 (0.25 mmol), NaN₃ (0.38 mmol) in EtOH (3 mL) at 55° C.
^{b} Isolated yield

### Example 4

### Cycloaddition Reaction Catalyzed by Using Nano Cu₂O to Form 3,5-Disubstituted Isoxazols

First, the first unsaturated compound and the second unsaturated compound are provided, the first unsaturated compound is oximes (as formula (3)), R3 is nitrobenzene, R4 is chlorine. The second unsaturated compound is alkynes, (as formula (1)), R1 is phenyl. Then, 50 mg, 0.25 mmol of the first unsaturated compound, 26 mg, 0.25 mmol of the second unsaturated compound and 75 ml, 0.75 mmol of Et₃N were placed in 3 ml of ethanol at 60° C. under nitrogen atmosphere to obtain 3,5-disubstituted isoxazols.

In order to detect the catalytic effect of various catalysts, cubes, octahedra (OC) or rhombic dodecahedra (RD) of the nano Cu₂O particles are added into the reaction, X-ray diffraction patterns thereof are shown in FIG. 2. The comparisons of reaction time/yield are shown in table 2 below. Rhombic dodecahedra (RD) of the nano Cu₂O particles catalyst reaches the fastest reaction rate in three reactions listed in Table 2.

TABLE 2
Comparison of reaction time and yield of forming
3,5-disubstituted isoxazols by cycloaddition reaction
catalyzed by different shapes of nano Cu₂O

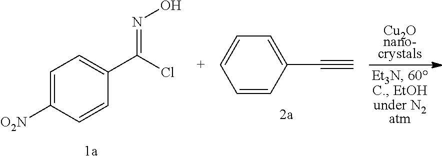

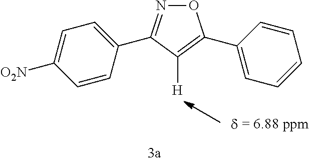 [SMILES: O=[N+]([O-])c1ccc(-c2cc(-c3ccccc3)on2)cc1]

| | | | Usage | | |
|---|---|---|---|---|---|
| Num- | | BET surface | amount | Time | |
| ber | Catalyst | area/m²g⁻¹ | (mg) | (h) | Yield^{b} |
| 1 | Cu₂O | 2.84 | 1 | 8 | 82 |
| | (cube) | | | | |
| 2 | Cu₂O | 0.56 | 5 | 6 | 89 |
| | (octahedra) | | | | |
| 3 | Cu₂O | 1.35 | 2 | 2.5 | 95 |
| | (rhombic | | | | |
| | dodecahedra) | | | | |

^{a} Reagents and conditions: 1a (50 mg, 0.25 mmol), 2a (26 mg, 0.25 mmol), Et₃N (75 mg, 0.75 mmol) EtOH (3 mL)
^{b}Isolated yield

As described above, if R4 of the first unsaturated compound is hydrogen, R3 may be various forms, and catalyzes the formation of 3,5-disubstituted isoxazols by rhombic dodecahedra nano Cu₂O particle. In the same condition, comparison to cubes or octahedral nano Cu₂O particle, using rhombic dodecahedra nano Cu₂O particle as catalyst has higher reaction rate and yield. The reactions are shown in table 3.

TABLE 3
Reaction time and yield of forming 3,5-disubstituted isoxazols catalyzed by rhombic dodecahedra nano Cu₂O particle

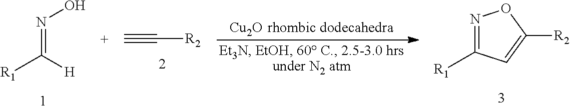

| Num- | | | | | Yield |
|---|---|---|---|---|---|
| ber | R1 | R2 | Product | Weight | % |
| 1 | 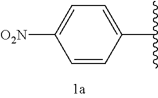 [SMILES: *c1ccc([N+](=O)[O-])cc1] | 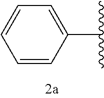 [SMILES: *c1ccccc1] | 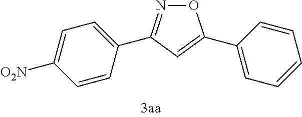 [SMILES: O=[N+]([O-])c1ccc(-c2cc(-c3ccccc3)on2)cc1] | 266 | 95 |
| 2 | 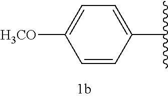 [SMILES: *c1ccc(OC)cc1] | 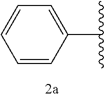 [SMILES: *c1ccccc1] | 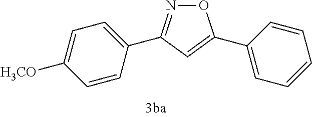 [SMILES: COc1ccc(-c2cc(-c3ccccc3)on2)cc1] | 251 | 91 |
| 3 | 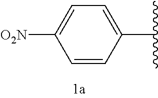 [SMILES: *c1ccc([N+](=O)[O-])cc1] | 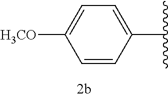 [SMILES: *c1ccc(OC)cc1] | 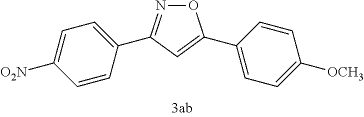 [SMILES: COc1ccc(-c2cc(-c3ccc([N+](=O)[O-])cc3)no2)cc1] | 296 | 92 |
| 4 | 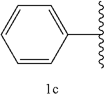 [SMILES: *c1ccccc1] | 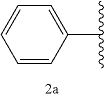 [SMILES: *c1ccccc1] | 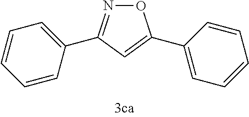 [SMILES: c1ccc(-c2cc(-c3ccccc3)on2)cc1] | 221 | 92 |
| 5 | 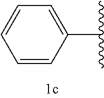 [SMILES: *c1ccccc1] | 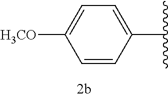 [SMILES: *c1ccc(OC)cc1] | 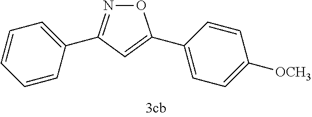 [SMILES: COc1ccc(-c2cc(-c3ccccc3)no2)cc1] | 251 | 90 |
| 6 | 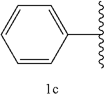 [SMILES: *c1ccccc1] | 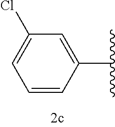 [SMILES: *c1cccc(Cl)c1] | 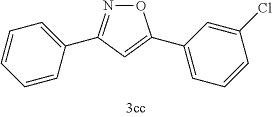 [SMILES: Clc1cccc(-c2cc(-c3ccccc3)no2)c1] | 255 | 89 |
| 7 | 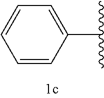 [SMILES: *c1ccccc1] | 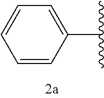 [SMILES: *c1ccccc1] | 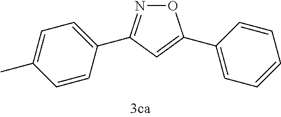 [SMILES: Cc1ccc(-c2cc(-c3ccccc3)on2)cc1] | 235 | 93 |
| 8 | 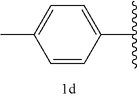 [SMILES: *c1ccc(C)cc1] | 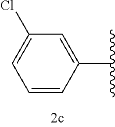 [SMILES: *c1cccc(Cl)c1] | 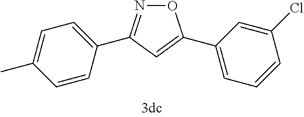 [SMILES: Cc1ccc(-c2cc(-c3cccc(Cl)c3)on2)cc1] | 269 | 90 |
| 9 | 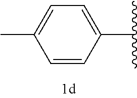 [SMILES: *c1ccc(C)cc1] | 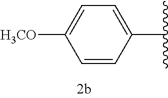 [SMILES: *c1ccc(OC)cc1] | 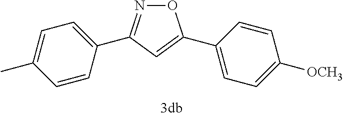 [SMILES: COc1ccc(-c2cc(-c3ccc(C)cc3)no2)cc1] | 265 | 91 |
| 10 | 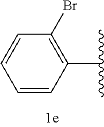 [SMILES: *c1ccccc1Br] | 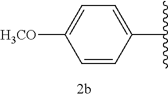 [SMILES: *c1ccc(OC)cc1] | 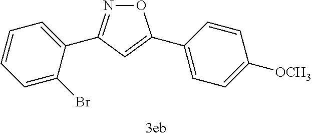 [SMILES: COc1ccc(-c2cc(-c3ccccc3Br)no2)cc1] | 330 | 95 |
| 11 | 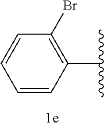 [SMILES: *c1ccccc1Br] | 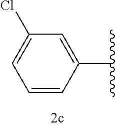 [SMILES: *c1cccc(Cl)c1] | 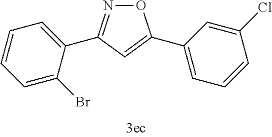 [SMILES: Clc1cccc(-c2cc(-c3ccccc3Br)no2)c1] | 334 | 94 |
| 12 | 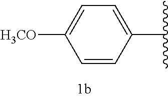 [SMILES: *c1ccc(OC)cc1] | 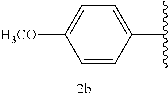 [SMILES: *c1ccc(OC)cc1] | 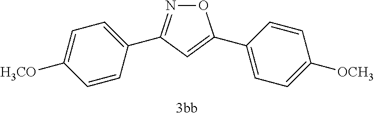 [SMILES: COc1ccc(-c2cc(-c3ccc(OC)cc3)on2)cc1] | 281 | 93 |
| 13 | 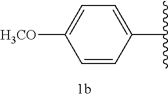 [SMILES: *c1ccc(OC)cc1] | 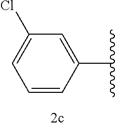 [SMILES: *c1cccc(Cl)c1] | 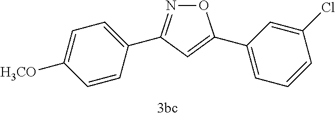 [SMILES: COc1ccc(-c2cc(-c3cccc(Cl)c3)on2)cc1] | 285 | 89 |
| 14 | 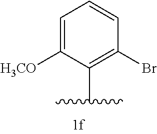 [SMILES: *c1c(Br)cccc1OC] | 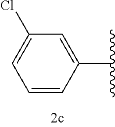 [SMILES: *c1cccc(Cl)c1] | 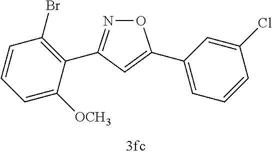 [SMILES: COc1cccc(Br)c1-c1cc(-c2cccc(Cl)c2)on1] | 364 | 87 |
| 15 | 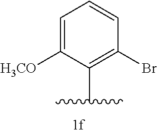 [SMILES: *c1c(Br)cccc1OC] | 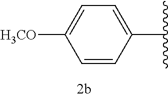 [SMILES: *c1ccc(OC)cc1] | 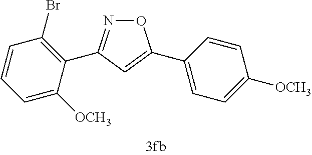 [SMILES: COc1ccc(-c2cc(-c3c(Br)cccc3OC)no2)cc1] | 360 | 90 |

### Example 5

### Synthesis of Cubes, Octahedra and Rhombic Dodecahedra of the Nano Gold Catalysts

The synthesis step of cubes, octahedra and rhombic dodecahedra of the nano gold catalysts are substantially the same, only need to change the volume of distilled water, ascorbic acid and solution of seed crystal while keep other parameters to be constant. Distilled water with different volume were added into each vials (for example, 9.550 mL for cube, 9.380 mL for octahedral and volume of distilled water for rhombic dodecahedra are as shown in table). Then, 10 μL, 0.01 M of sodium bromine was introduced to grow nano cubes and dodecahedra. To obtain octahedra, 50 μL, 0.001 M of potassium iodide was added, instead of sodium bromine. Finally, 90, 220 and 150 μL, 0.04 M of ascorbic acid solution, were added, respectively, to synthesize nano cubes, octahedra and rhombic dodecahedra. Total volume of each vial is 10 ml. Next, 100 μL or different volumes of solution of seed crystal were added into vial A and shaken until color of the solution becomes light pink (˜3 sec.). Then, 100 μL of solution in vial A was transferred into vial B and fully mix-10 s. Standing the solution in vial B for 15 min to grow the grain and centrifuging at 9500 rpm for 10 min three times.

TABLE 4
Synthesis of cube (CU31), octahedral (OC33) and rhombic dodecahedra
(RD78, RD53, RD42, RD32) of the nano gold catalysts

| | | | | | | Seed |
|---|---|---|---|---|---|---|
| | | | 0.01M | 0.01M | 0.04M | crystal |
| | CTAC | H₂O | AuCl₄ | NaBr | AA | solution |
| Sample | (g) | (μL) | (μL) | (μL) | (μL) | (μL) |
| RD78 | 0.32 | 9965 | 250 | 10 | 150 | 25 |
| RD53 | | 9545 | | | | 45 |
| RD42 | | 9515 | | | | 75 |
| RD32 | | 9490 | | | | 100 |
| CU31 | | 9550 | | | 90 | 100 |
| OC33 | | 9380 | | 0.001M | 220 | 100 |
| | | | | KI50 μL | | |

### Example 6

### Catalyze Cycloaddition Reaction Using Nano Gold to Form 1,2,3-Triazoles

First, the first unsaturated compound and the second unsaturated compound were prepared, the first unsaturated compound is alkynes, as shown in formula (1), R1 is phenyl. The second unsaturated compound is azides, as shown in formula (2), R2 is phenyl. The second unsaturated compound may pre-synthesize by azide salts NaN₃ and halides R2-X. Next, 0.25 mmol of the first unsaturated compound and 0.56 mmol of Et₃N were added into water, and vibrated and stirred in room temperature for 15 min, then the second unsaturated compound was added, and heat at 60° C. to obtain 1,2,3-Triazoles.

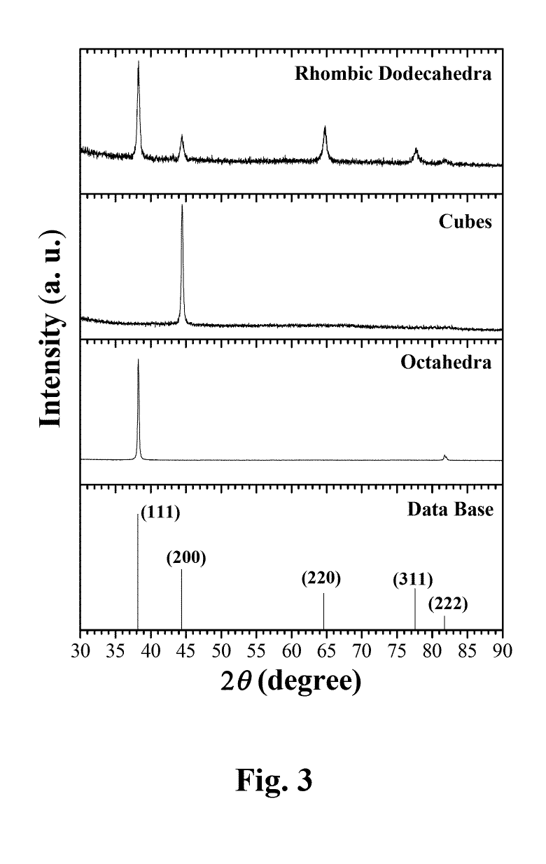

In order to detect the catalytic effect of various catalysts, cube (CU31), octahedral (OC33) or rhombic dodecahedra (RD78, RD53, RD42, RD32) of the nano gold particles. FIG. 3 shows X-ray diffraction patterns of nano gold particle of the present invention, in which cube mainly includes {100} crystal plane, octahedral mainly includes {111} crystal plane, and rhombic dodecahedra include {110} crystal plane. Herein, cube and octahedral are perfect cube and octahedral, so as they almost have no {110} crystal plane. The comparison of the isolated yield in the same reaction time are shown in table 4 below, wherein rhombic dodecahedra nano gold particle catalyst may obtain the highest isolated yield and regioselectivity in the same time, and it may attribute to {110} crystal plane.

TABLE 5
Comparison of reaction time and yield of forming 1,2,3-Triazoles by cyclo-
addition reaction catalyzed by different shapes of nano gold particles

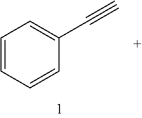

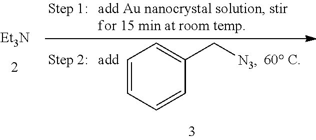

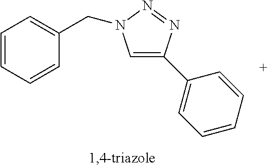 [SMILES: c1ccc(Cn2cc(-c3ccccc3)nn2)cc1]

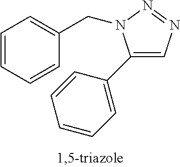 [SMILES: c1ccc(Cn2nncc2-c2ccccc2)cc1]

| | | | Isolated | | |
|---|---|---|---|---|---|
| | Size | Time | yield | Regioselectivity^{b} | TOF |
| Catalyst | (nm) | (h) | (%) | 1,4:1,5 | (h⁻¹) |
| RD78 | 78 | 6 | 20 | 100:0 | 40.0 |
| RD53 | 53 | 6 | 35 | 100:0 | 70.0 |
| RD42 | 42 | 6 | 49 | 100:0 | 96.7 |
| RD32 | 32 | 6 | 72 | 100:0 | 144.3 |
| CU31 | 31 | 6 | 44 | 66:34 | 62.2 |
| OC33 | 33 | 6 | 32 | 52:48 | 39.2 |

^{a} Reagents and conditions: 1 (0.25 mmol), 2 (0.25 mmol) in H₂O at 60° C.
^{b}Regioselecitivity calculated from ¹H-NMR analysis of crude reaction mixture.

As described above, R1 of the first unsaturated compound and R2 of the second unsaturated compound may have other forms, and catalyzes the formation of 1,2,3-Triazoles by rhombic dodecahedra nano gold particles. In the same condition, comparison to cubes or octahedral nano gold particles in table 4, using rhombic dodecahedra nano gold particles as catalyst has higher reaction rate and yield. The reactions are shown in table 5.

TABLE 6
Comparison of reaction time and yield of forming 1,2,3-Triazoles by
cycloaddition reaction catalyzed by rhombic dodecahedra nano gold particles

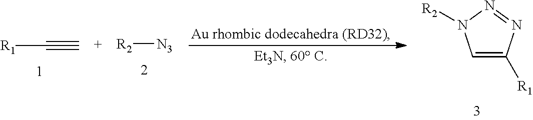

| Num- | | Organic | | | Time | Yield |
|---|---|---|---|---|---|---|
| ber | Alkynes | Halides | Product | Weight | (h)^{b} | %^{c} |
| 1 | 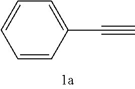 | 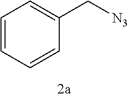 | 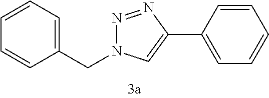 [SMILES: c1ccc(Cn2cc(-c3ccccc3)nn2)cc1] | 235 | 6 | 72 |
| 2 | 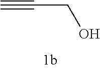 | 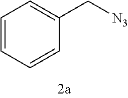 | 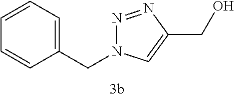 [SMILES: OCc1cn(Cc2ccccc2)nn1] | 189 | 6 | 71 |
| 3 | ≡—SiMe₃ 1c | 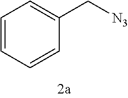 | 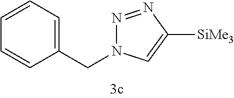 [SMILES: C[Si](C)(C)c1cn(Cc2ccccc2)nn1] | 231 | 5 | 71 |
| 4 | 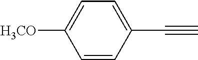 | 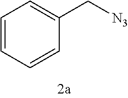 | 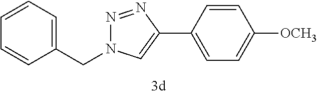 [SMILES: COc1ccc(-c2cn(Cc3ccccc3)nn2)cc1] | 265 | 5.5 | 67 |
| 5 | 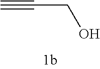 | 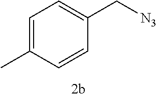 | 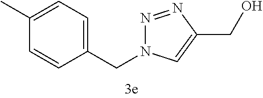 | 203 | 6 | 62 |

^{a} Reagents and conditions: 1 (0.25 mmol), 2 (0.25 mmol), Et₃N (0.56 mmol) in H2O at 60° C.
^{b}LRMS is detect by EI ionization source
^{c}Isolated yield

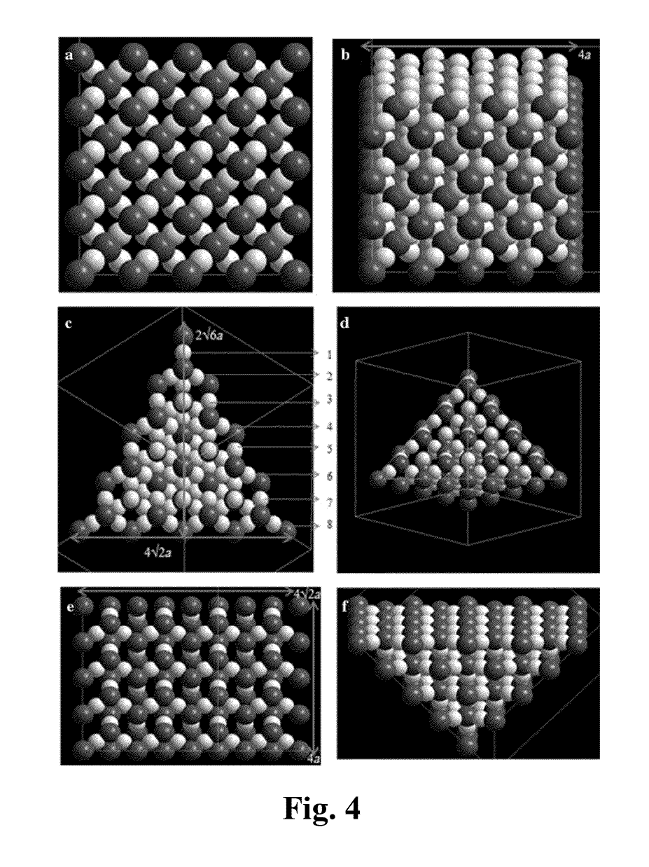

The result of the present invention may be explained by the analysis of different crystal planes of Cu₂O. FIG. 4 shows the crystal models of {100}, {110} and {111} crystal planes of Cu₂O. {100} crystal plane includes surface planes of a body-centered cubic unit cell of Cu₂O, wherein oxygen atoms form the crystal lattice and copper atoms occupy half of the tetrahedral sites. However, {100} crystal plane may also be present to expose terminal Cu atoms. For consistency with experimental observations of the low reactivity of nanocubes, the surface Cu atoms are considered to lie just below the uppermost layer of oxygen atoms. The {111} crystal plane contains terminal copper and oxygen atoms. However, many of the surface Cu atoms reside below the plane of surface oxygen atoms (shown in FIG. 3c). {110} crystal plane is terminated with copper and oxygen atoms lying essentially on the same plane, and so as all the surface Cu atoms are fully exposed (shown as FIG. 3f).

An area density analysis of surface Cu atoms reveals that the {110} crystal plane actually has the lowest surface Cu atom density (10.98, 14.27, and 7.76 Cu atoms/nm² for the {100}, {111}, and {110} crystal planes of Cu₂O, respectively). However, all of the surface Cu atoms on the {110} crystal planes are fully exposed for interaction with ligands, whereas many of the surface Cu atoms of the {111} crystal plane are partially exposed and only partially exposed Cu atoms are available for the {100} crystal plane to hinder the ligand interaction. These differences explain the observed relative catalytic activity of these surfaces.

In summary, the present invention provides a metal or metal oxide catalyst with {110} crystal plane to increase the adsorption sites for reactant, evaluate reactivity and improve the reaction rate of cycloaddition reaction, so as to obtain higher yield. In addition, the present invention also provides a catalyst mentioned above to complete a catalytic reaction and evaluate reactivity and improve the reaction rate.

The embodiments as above only illustrate the technical concepts and characteristics of the present invention; it is purposed for person ordinary skill in the art to understand and implement the present invention, but not for the limitation to claims of the present invention. That is, any equivalent change or modification in accordance with the spirit of the present invention should be covered by the appended claims.

What is claimed is:

1. A catalytic reaction, comprising:
providing a catalyst, wherein the catalyst is metal or metal oxide nanoparticles and at least comprises {110} crystal planes;
providing a first unsaturated compound and a second unsaturated compound; and
providing the catalyst to perform a cycloaddition reaction of the first unsaturated compound and the second unsaturated compound and obtain a product.

2. The catalytic reaction as claimed in claim 1, wherein the catalyst is cubes or octahedra and comprise a cut edge structure to expose the {110} crystal planes.

3. The catalytic reaction as claimed in claim 1, wherein the catalyst is rhombic dodecahedra.

4. The catalytic reaction as claimed in claim 1, wherein the catalyst is made of cuprous oxide or gold.

5. The catalytic reaction as claimed in claim 1, wherein a particle size of the catalyst ranges from 30-300 nm.

6. The catalytic reaction as claimed in claim 1, wherein the product is a heterocyclic compound.

7. The catalytic reaction as claimed in claim 1, wherein the product is a cyclic compound.

8. The catalytic reaction as claimed in claim 1, wherein the product includes triazole or isoxazoles.

9. The catalytic reaction as claimed in claim 1, wherein the catalyst has a regioselectivity greater than 50%.

10. The catalytic reaction as claimed in claim 1, wherein the cycloaddition reaction includes [2+2] cycloaddition reaction, [2+3] cycloaddition reaction, [3+2] cycloaddition reaction, [4+2] cycloaddition reaction, [4+3] cycloaddition reaction or [6+4] cycloaddition reaction.

11. The catalytic reaction as claimed in claim 1, wherein the cycloaddition reaction incldues Diels-Alder reaction, Huisgen cycloaddition reaction or Nitrone-olefin cycloaddition reaction.

12. The catalytic reaction as claimed in claim 1, wherein each of the first unsaturated compound and the second unsaturated compound is selected from a group consisting of alkenes, alkynes and 1,3 dipolar compound.

13. The catalytic reaction as claimed in claim 12, wherein the alkynes is represented by formula (1): R₁ [symbol] . . . (1), wherein R1 is independently selected from a group consisting of hydroxyl, carboxyl, ester, nitro, alkyl silicon, substituted or unsubstituted C1-C10 alkyl, substituted or unsubstituted C2-C10 alkenyl, substituted or unsubstituted C2-C10 alkynyl, substituted or unsubstituted C3-C20 cycloalkyl, substituted or unsubstituted C3-C20 cycloalkenyl, substituted or unsubstituted C1-C20 heterocycloalkyl, substituted or unsubstituted C1-C20 heterocycloalkenyl, substituted or unsubstituted aryl, and substituted or unsubstituted heteroaryl.

14. The catalytic reaction as claimed in claim 12, wherein the 1,3 dipolar compound comprises azides, wherein the azides is represented by formula (2): R₂—N₃ . . . (2), wherein R2 is selected from a group consisting of hydroxyl, carboxyl, ester, nitro, alkyl silicon, substituted or un substituted C1-C10 alkyl, substituted or unsubstituted C2-C10 alkenyl, substituted or unsubstituted C2-C10 alkynyl, substituted or unsubstituted C3-C20 cycloalkyl, substituted or unsubstituted C3-C20 cycloalkenyl, substituted or unsubstituted C1-C20 heterocycloalkyl, substituted or unsubstituted C1-C20 heterocycloalkenyl, substituted or unsubstituted aryl, and substituted or unsubstituted heteroaryl.

15. The catalytic reaction as claimed in claim 12, wherein the 1,3 dipolar compound comprises oximes, wherein the oximes is represented by formula (3): 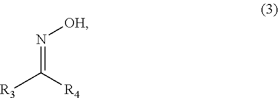
wherein R3 is independently selected from a group consisting of hydroxyl, carboxyl, ester, nitro, alkyl silicon, substituted or un substituted C1-C10 alkyl, substituted or unsubstituted C2-C10 alkenyl, substituted or unsubstituted C2-C10 alkynyl, substituted or unsubstituted C3-C20 cycloalkyl, substituted or unsubstituted C3-C20 cycloalkenyl, substituted or unsubstituted C1-C20 heterocycloalkyl, substituted or unsubstituted C1-C20 heterocycloalkenyl, substituted or unsubstituted aryl, and substituted or unsubstituted heteroaryl; R4 is selected from hydrogen and halogen.

16. The catalytic reaction as claimed in claim 8, wherein the product includes triazole represented by formula (4): 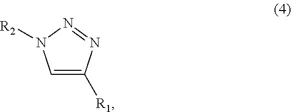
wherein R1 and R2 are independently selected from a group consisting of hydroxyl, carboxyl, ester, nitro, alkyl silicon, substituted or un substituted C1-C10 alkyl, substituted or unsubstituted C2-C10 alkenyl, substituted or unsubstituted C2-C10 alkynyl, substituted or unsubstituted C3-C20 cycloalkyl, substituted or unsubstituted C3-C20 cycloalkenyl, substituted or unsubstituted C1-C20 heterocycloalkyl, substituted or unsubstituted C1-C20 heterocycloalkenyl, substituted or unsubstituted aryl, and substituted or unsubstituted heteroaryl.

17. The catalytic reaction as claimed in claim 15, wherein the product includes isoxazole represented by formula (5): 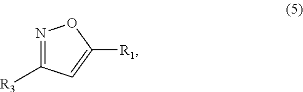
wherein R1 and R3 are independently selected from a group consisting of hydroxyl, carboxyl, ester, nitro, alkyl silicon, substituted or un substituted C1-C10 alkyl, substituted or unsubstituted C2-C10 alkenyl, substituted or unsubstituted C2-C10 alkynyl, substituted or unsubstituted C3-C20 cycloalkyl, substituted or unsubstituted C3-C20 cycloalkenyl, substituted or unsubstituted C1-C20 heterocycloalkyl, substituted or unsubstituted C1-C20 heterocycloalkenyl, substituted or unsubstituted aryl, and substituted or unsubstituted heteroaryl.
